(19) United States
(12) Patent Application Publication
(10) Pub. No.: US 2007/0281303 A1
(43) Pub. Date: Dec. 6, 2007

(54) DNA STORAGE AND DISPLAY VESSEL AND METHOD

(75) Inventors: Andrew Patrick Isakson, Gibsonville, NC (US); Brian Walter Meehan, Elon, NC (US)

Correspondence Address: ADAMS EVANS P.A., Suite 2350 Charlotte Plaza, 201 South College Street, CHARLOTTE, NC, 28244, US

(73) Assignee: DNA SECURITY, INC., Burlington, NC (US)

(21) Appl. No.: 11/422,154

(22) Filed: Jun. 5, 2006

(51) Int. Cl. C12Q 1/68; C12M 3/00

(52) U.S. Cl. 435/6; 435/287.2

(57) ABSTRACT

A storage display vessel for biological material, including a block of, for example, glass or plastic, having at least one transparent area, and a void defining a storage area in the transparent area of the block communicating with an outside surface of the block for receiving biological material therein for storage. A plug is provided, and is adapted for sealing the storage area and the biological material therein from communication with the outside surface of the block. At least one three-dimensional internal visual indicator is carried by the block indicating that the block contains a biological material.

### TECHNICAL FIELD AND BACKGROUND OF THE INVENTION

This invention relates to a storage and display container, or vessel, for permanent storage of a biological material such as deoxyribonucleic acid (DNA), and a related method. DNA is usually in the form of a double helix that contains the genetic instructions specifying the biological development of all cellular forms of life, and most viruses. DNA is a long polymer of nucleotides and encodes the sequence of the amino acid residues in proteins using the genetic code, a triplet code of nucleotides.

DNA is often referred to as the molecule of heredity as it is responsible for the genetic propagation of most inherited traits. In humans, these traits can range from hair color to disease susceptibility. During cell division, DNA is replicated and can be transmitted to offspring during reproduction. Lineage studies can be done based on the facts that the mitochondrial DNA only comes from the mother, and the male Y chromosome only comes from the father.

Every person's DNA, their genome, is inherited from both parents. The mother's mitochondrial DNA together with twenty-three chromosomes from each parent combine to form the genome of a zygote, the fertilized egg. As a result, with certain exceptions such as red blood cells, most human cells contain 23 pairs of chromosomes, together with mitochondrial DNA inherited from the mother.

These facts have resulted in the use of DNA as a forensic tool for many purposes, including crime investigation, family and paternal determinations, and disease diagnosis and treatment. DNA is presently often stored in a haphazard manner that may leave it subject to contamination or deterioration. At the same time, some individuals view DNA as a symbol of familial connection and history, and would like to themselves maintain the DNA of family and loved-ones. The present invention provides a vessel where DNA can be safely stored for an indefinite period of time, and easily accessed if future circumstances require use of the DNA for any purpose.

### SUMMARY OF THE INVENTION

Therefore, it is an object of the invention to provide a storage container, or vessel, for permanent storage of DNA.

It is another object to provide a vessel for storage of DNA that allows access to the DNA if future circumstances require.

It is another object to provide a vessel for storage of DNA that provides for the display of the DNA while stored.

These and other objects and advantages of the invention are disclosed herein and include a storage display vessel for biological material, comprising a block, which may be plastic, glass, or other suitable materials, having at least one transparent area, and a void defining a storage area in the transparent area of the block communicating with an outside surface of the block for receiving biological material therein for storage. A plug is provided, and is adapted for sealing the storage area and the biological material therein from communication with the outside surface of the block. At least one visual indicator is carried by the block indicating that the block contains a biological material.

According to one embodiment of the invention, the block is formed of transparent acrylic resin.

According to another embodiment of the invention, the void is elongate, and wherein the plug has a length shorter than the void.

According to yet another embodiment of the invention, the at least one visual indicator carried by the block comprises a thermoplastic material having a melting temperature whereby a pattern is formed in the interior of the block by the interaction of at least two laser beams melting the thermoplastic material at points of intersection of the two laser beams according to a predetermined pattern design.

According to another embodiment of the invention, the design comprises a visual representation of a DNA double helix.

According to another embodiment of the invention, the plug is sealed into the void using a transparent glue.

According to another embodiment of the invention, the plug includes a recess in an interior end thereof that mates with the storage area.

According to another embodiment of the invention, the storage display vessel is in combination with a biological material carried on the surface of a carrier body.

According to another embodiment of the invention, the biological material comprises DNA.

According to another embodiment of the invention, the biological material comprises DNA and the carrier body comprises a plurality of beads onto which the DNA is dried.

According to another embodiment of the invention, the beads comprise glass beads.

According to another embodiment of the invention, the void is formed by a method selected from the group consisting of drilling and casting.

According to another embodiment of the invention, a method is provided for storing and displaying biological material, comprising the steps of forming a plastic block having at least one transparent area, and forming a void in the transparent area of the block communicating with an outside surface of the block for receiving biological material therein for storage. A biological material is inserted into the void, and the void and the biological material is sealed therein from communication with the outside surface of the block. At least one visual indicator is carried by the block for indicating that the block contains a biological material.

According to another embodiment of the invention, the method includes the step of sealing the void with a plug that is shorter than the void.

According to another embodiment of the invention, the method includes the steps of drying the biological material onto a carrier body and inserting the carrier body and biological material carried thereon into the void.

According to another embodiment of the invention, the method includes the steps of drying the biological material onto a plurality of glass beads, and inserting the glass beads and the biological material carried thereon into the void.

According to another embodiment of the invention, the biological material comprises DNA.

According to another embodiment of the invention, the step of providing the visual indicator comprises the steps of forming the block of a thermoplastic material having a melting temperature whereby a pattern is formed in the interior of the block by the interaction of at least two laser beams melting the thermoplastic material at points of intersection of the two laser beams according to a predetermined pattern design. According to another embodiment of the invention, the design comprises a visual representation of a DNA double helix.

According to another embodiment of the invention, the method includes forming in the block a series of labels and numbers that represent the locations on the human genome helix of the DNA.

### BRIEF DESCRIPTION OF THE DRAWINGS

Some of the objects of the invention have been set forth above. Other objects and advantages of the invention will appear as the description of the invention proceeds when taken in conjunction with the following drawings, in which:

FIG. 1 is a perspective view of a storage display vessel for biological material according to an embodiment of the invention;

FIG. 2 is a front elevation of the storage display vessel shown in FIG. 1;

FIG. 3 is a side elevation of the storage display vessel shown in FIG. 1;

FIG. 4 is a perspective view of the storage display vessel of FIGS. 1-3 showing one preferred location for the void, and plug insertion;

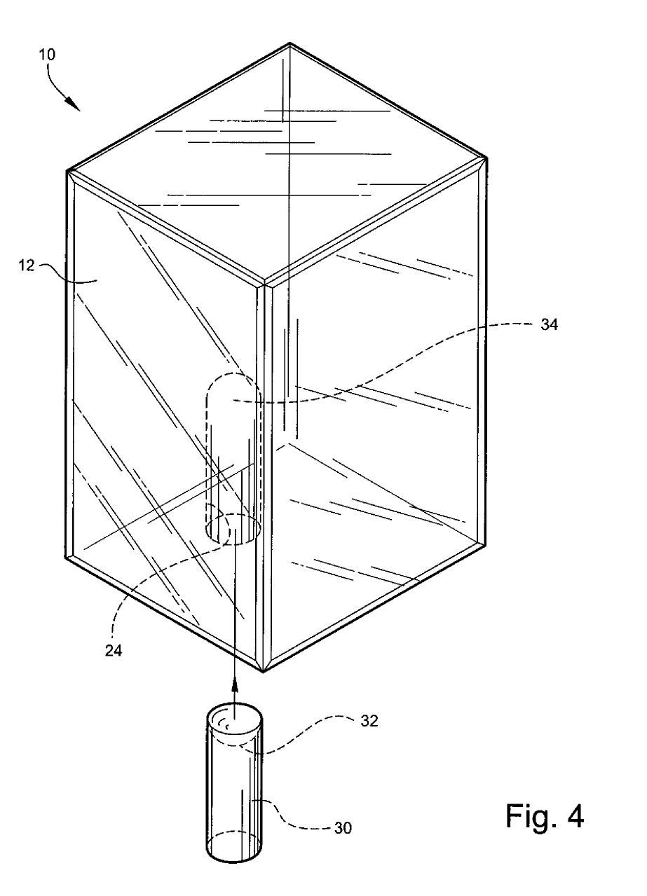

FIG. 5 is a fragmentary side elevational cross-section of the storage vessel as shown in FIG. 3, showing details of the void and plug; and

FIG. 6 is a flow chart of the method according to an embodiment of the invention.

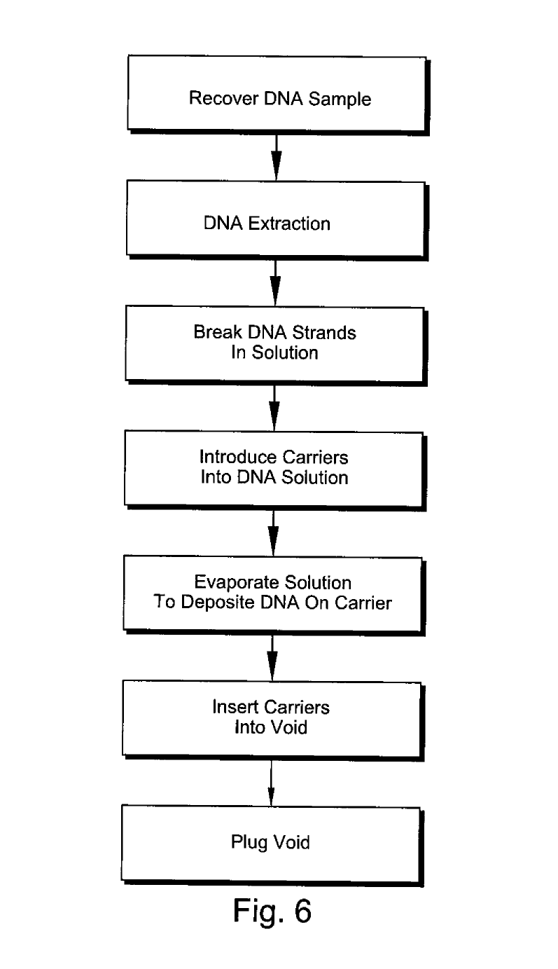

### DESCRIPTION OF THE PREFERRED EMBODIMENTS AND BEST MODE

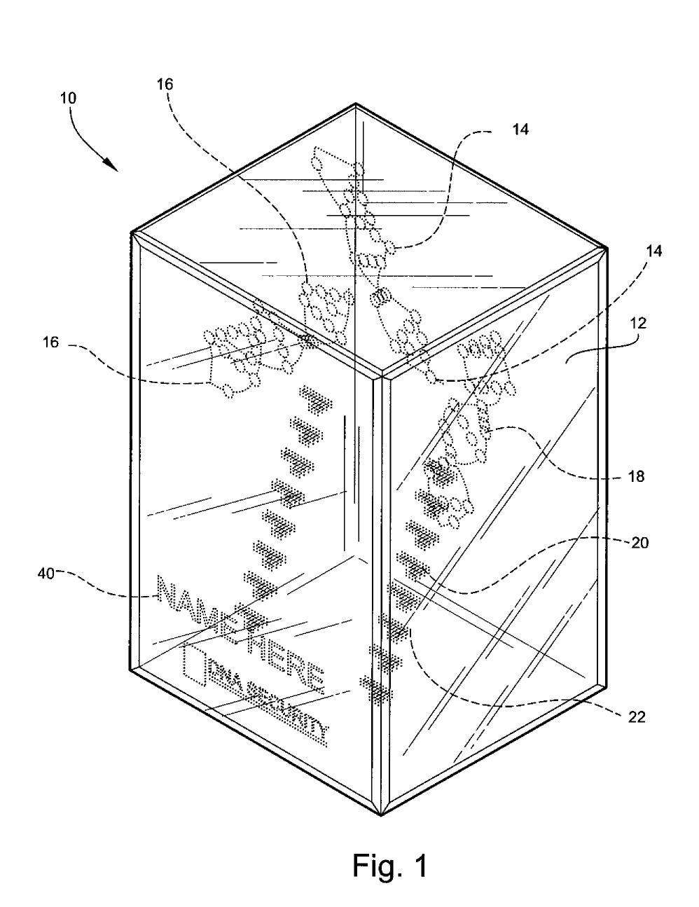

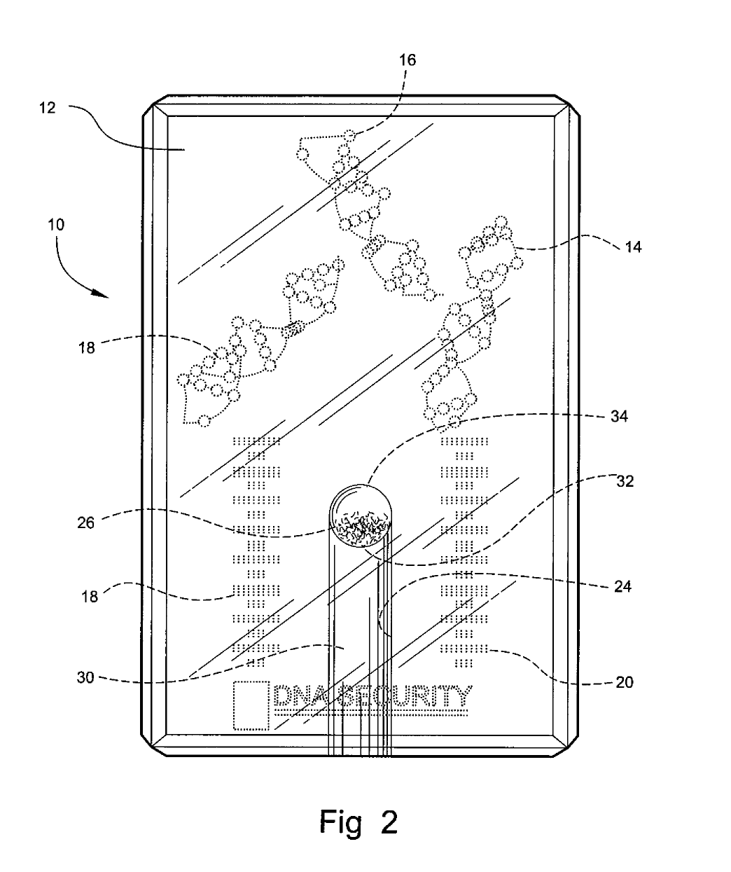

Referring now specifically to the drawings, a storage vessel for storing and displaying a biological material such as DNA according to the present invention is shown generally in FIGS. 1-3 at reference numeral 10. The vessel 10 according to the preferred embodiment described in this application comprises a block 12 of acrylic cast in a mold to form a transparent “crystal” that serves as a blank out of which the finished product will be fabricated. The refractive qualities of the acrylic block are such that the images formed within the block, as explained below, are simultaneously visible from different aspects, as shown in FIG. 1. Alternatively, the block 12 may be formed from a glass material, or any other material that can be fabricated to contain the DNA material in a manner described or suggested in this application, for example, a transparent or translucent material, or a material that can be laser-etched to form an interior pattern in the manner described or suggested below.

According to a preferred embodiment, the vessel includes storage and display elements. Specifically, a three dimensional representation of several strand segments 14, 16, 18 of DNA are laser-formed in the molded acrylic block. The laser forming process includes the use of a computer in which is stored a predetermined pattern design, such as the DNA strand segments 14, 16, 18. Two lasers are aimed at the block 12 from different positions, and the laser beams simultaneously “trace” the pattern. The melting temperature of the acrylic is such that neither laser alone will generate sufficient heat to melt or deform the acrylic, but the collective power of both lasers will melt the acrylic. Thus, at the points where the two laser beams intersect the acrylic melts to form a small, translucent, frosted spot in the block 12. The pattern of spots thus formed define the designs of the DNA segments 14, 16, 18, engraved in the interior of the block 12. This overall process is known in the art and is not, per se, novel.

The same process may be used to engrave other graphical elements into the block. As shown in FIGS. 1-3, stair stepped arrays of information 20, 22 may comprise any desired data or graphical material. In the embodiment shown in the Figures, the arrays of information comprise lists of locations on the human genome helix where the DNA sampling was carried out, and numbers representing the DNA test data-one set of numbers from the father, and one set of numbers from the mother.

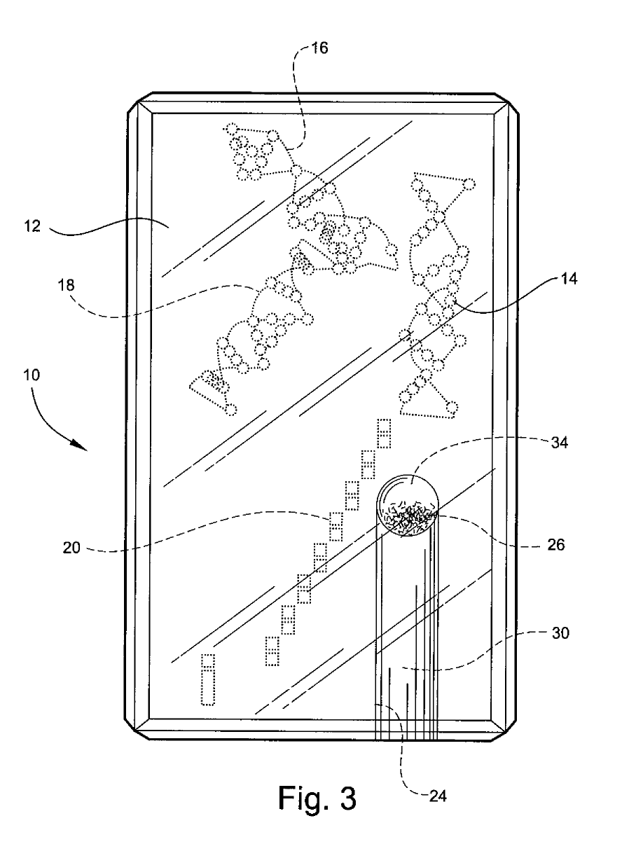

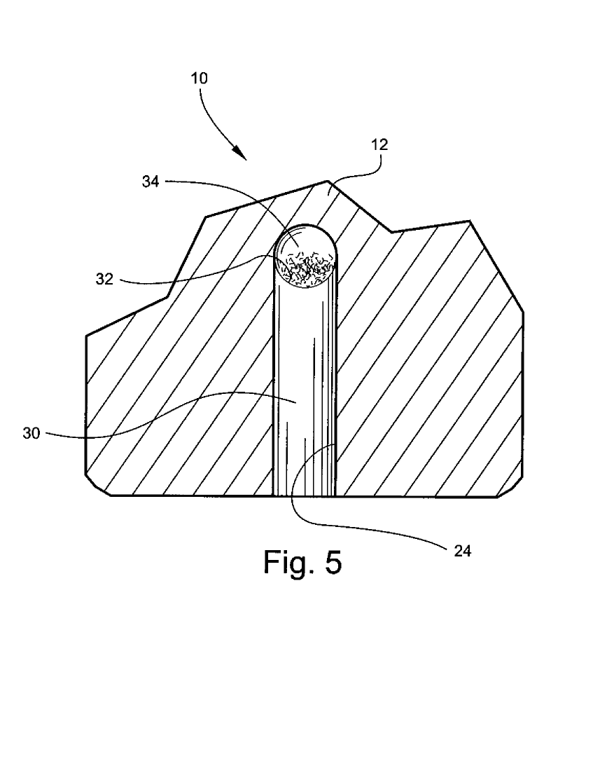

An elongate void 24 is formed in the block 12 by drilling or casting and is used to store a sample of a person's DNA. Preferably, the inferior walls of the void are burnished to provide a smooth, completely transparent surface finish. In accordance with a preferred embodiment of the invention, the DNA is applied to and carried on the surface of a plurality of small objects, such as beads 26. The beads 26 with the DNA on the surface are placed in the void 24, and then a plug 30 formed of the same clear acrylic is inserted into the void and secured therein with an acrylic glue of the type that dries clear. Ideally, the plug 30 closely conforms to the size and shape of the sidewalls of the void 24 and becomes essentially invisible. As shown particularly in FIGS. 3 and 5, the plug 30 is shorter than the length of the void 24 such that a interior storage area 34 remains after insertion of the plug 30, in which the beads 26 are stored. In addition, the plug 30 in accordance with one embodiment of the invention has a concavity 32 formed on its end that forms a portion of the storage area 34.

While stored, the beads 26 are visible and can be observed through the transparent acrylic material of the block 12. Unless exposed to heat of sufficient intensity and duration to melt the acrylic material through to the storage area 34, the DNA will be preserved indefinitely. If for whatever reason the DNA must be retrieved, the storage area may be penetrated with a drill and one or more of the beads 26 removed. The block 12 may thereafter be resealed in the manner described above.

DNA does not exist as a free molecule in a cell, but instead is associated with proteins and RNA. Thus, the process of extracting and isolating DNA from a cell and other molecules is the first step for many laboratory procedures in biotechnology. This process involves gently breaking up the cells, denaturing the proteins, and then precipitating the DNA as a white fibrous material. The exact steps involved in this process vary depending on the organism from which the DNA will be extracted. The procedure used according to the practice of this invention may be any known method of DNA extraction and isolation, for example, the Marmur preparation, which is used worldwide in biotechnology laboratories. See, Marmur, “A Procedure for the Isolation of Deoxyribonucleic Acid from Micro-organisms,” J. Mol. Biol., vol. 3, pp. 208-218 (1961).

DNA may be obtained by numerous means such as, for example, obtaining cells from inside the mouth and cheek by means of a swab or scraping tool. Once the DNA has been extracted and isolated, a solution containing the DNA is prepared and a plurality of carrier bodies, such as the beads 26, are placed in the solution. The DNA is then dried by evaporating the solution. As evaporation takes place, the DNA is precipitated out of the solution onto the surface of the beads 26. The beads 26 may be smooth, or may have a rough surface texture. The rough surface will provide a greater overall surface area on which the DNA may be deposited if the quantity of DNA to be deposited on the beads is important. The beads 26 are then ready for insertion into the void 24.

As noted above, any biological sample may be prepared and stored in the void 24 of the block 12. Human DNA may be stored for reasons of both preservation and remembrance. Similarly, DNA from animal pets, race horses or other animals may be processed and stored. As shown in FIG. 1, a name 40 or other identifier may be three-dimensionally engraved into the block 12 as described above. Any other image, such as a portrait of the person or animal whose DNA is stored in the storage area 34 may also be engraved into the block 12.

The block 12 may be any size or shape, and can serve as an insert in a necklace, grave marker, or other structure.

A storage and display container, or vessel, for permanent storage of a biological material such as deoxyribonucleic acid (DNA), and a related method, is described above. Various details of the invention may be changed without departing from the scope of the invention. Furthermore, the foregoing description of the preferred embodiment of the invention and best mode for practicing the invention are provided for the purpose of illustration only and not for the purpose of limitation, the invention being defined by the claims.

We claim:

1. A storage display vessel for biological material, comprising:
(a) a block having at least one transparent area:
(b) a void defining a storage area in the transparent area of the block communicating with an outside surface of the block for receiving biological material therein for storage;
(c) a plug adapted for sealing the storage area and the biological material therein from communication with the outside surface of the block; and
(d) at least one three-dimensional, internal visual indicator carried by the block indicating that the block contains a biological material.

2. A storage display vessel according to claim 1, wherein the block is formed of a group consisting of transparent acrylic resin or glass.

3. A storage display vessel according to claim 1, wherein the void is elongate, and wherein the plug has a length shorter than the void.

4. A storage display vessel according to claim 1, wherein the at least one visual indicator carried by the block comprises a thermoplastic material having a melting temperature whereby a pattern is formed in the interior of the block by the interaction of at least two laser beams melting the thermoplastic material at points of intersection of the two laser beams according to a predetermined pattern design.

5. A storage display vessel according to claim 1, wherein the design comprises a visual representation of a DNA double helix.

6. A storage display vessel according to claim 1, wherein the plug is sealed into the void using a transparent glue.

7. A storage display vessel according to claim 1, wherein the plug includes a recess in an interior end thereof that mates with the storage area.

8. A storage display vessel according to claim 1, in combination with a biological material carried on the surface of a carrier body.

9. A storage display vessel according to claim 8, wherein the biological material comprises DNA.

10. A storage display vessel according to claim 8, wherein the biological material comprises DNA and the carrier body comprises a plurality of beads onto which the DNA is dried.

11. A storage display vessel according to claim 10, wherein the beads comprise glass beads.

12. A storage display vessel according to claim 1, wherein the void is formed by a method selected from the group consisting of drilling and casting.

13. A method of storing and displaying biological material, comprising the steps of:
(a) forming a block having at least one transparent area:
(b) forming a void in the transparent area of the block communicating with an outside surface of the block for receiving biological material therein for storage;
(c) inserting biological material into the void;
(d) sealing the void and the biological material therein from communication with the outside surface of the block; and
(e) providing at least one three-dimensional, internal visual indicator carried by the block for indicating that the block contains a biological material.

14. A method according to claim 13, wherein the step of forming the block comprises the step of forming a block of a material selected from the group consisting of a thermoplastic material or a glass material.

15. A method according to claim 13, and including the steps of drying the biological material onto a carrier and inserting the carrier and biological material carried thereon into the void.

16. A method according to claim 13, and including the steps of drying the biological material onto a plurality of glass beads, and inserting the glass beads and the biological material carried thereon into the void.

17. A method according to claim 13, wherein the biological material comprises DNA.

18. A method according to claim 13, and the step of providing the visual indicator comprises the steps of forming the block of a material having a melting temperature whereby a pattern is formed in the interior of the block by the interaction of at least two laser beams melting the material at points of intersection of the two laser beams according to a predetermined pattern design.

19. A method according to claim 18, wherein the design comprises a visual representation of a DNA double helix.

20. A method according to claim 19, wherein the step includes forming in the block a series of labels and numbers that represent the locations on the human genome helix of the DNA.
